(19) United States
(12) Patent Application Publication
(10) Pub. No.: US 2005/0075560 A1
(43) Pub. Date: Apr. 7, 2005

(54) Stereotactic frame and method for supporting a stereotactic frame

(76) Inventors: Henri Hannula, Helsinki (FI); Matti Aho, Espoo (FI)

Correspondence Address: BIRCH STEWART KOLASCH & BIRCH, PO BOX 747, FALLS CHURCH, VA, 22040-0747, US

(21) Appl. No.: 10/627,867

(22) Filed: Jul. 28, 2003

(30) Foreign Application Priority Data
Jul. 29, 2002 (FI) 20021416

(51) Int. Cl. A61B 5/05

(52) U.S. Cl. 600/424

(57) ABSTRACT

A stereotactic frame (1) is disclosed in this publication. The stereotactic frame (1) is adapted for the localization of organs in conjunction with the examination and/or treatment operations performed thereon. The stereotactic frame (1) according to the invention has been developed to give enhanced freedom of movement for the subject being examined and/or treated. The publication also discloses a method for supporting a stereotactic frame.

The invention relates to a stereotactic frame according to the preamble of claim 1.

The invention also relates to a method according to the preamble of claim 13 for supporting a stereotactic frame.

This kind of stereotactic frame is used in examination and/or therapy on the nervous system, muscles or other organs of a human or animal patient with the help of an analysis method based on accurate localization. Hence, the present invention is most appropriately utilized in the examination and/or treatment of the head and brain.

Biological tissue such as the brain can be stimulated by imposing an electric field thereon. In transcranial magnetic stimulation (TMS), the electric field is generated with the help of a varying magnetic field. In this fashion, magnetic stimulation may be utilized for the stimulation of the human brain, peripheral nervous system or muscles. The magnetic field is invoked by a coil excited with a strong current pulse of short duration. As a result, the coil is surrounded by a magnetic field whose strength decays rapidly at a greater distance from the coil. By the same token decreases the stimulating effect of the magnetic field on biological tissue. In conjunction with magnetic stimulation, it is typical that even so small a shift as 5 to 10 mm from the nominal position of the coil or a 10° tilt angle of the coil can change the stimulation effect on the target as much as 50%. The stimulation point is registered with the help of a localization system. After the location registration step, the system controls the stimulation coil on the head into a position wherein the coil evokes most effectively the stimulus on the desired target point in the brain. Depending on the alignment accuracy of the magnetic field generated by the coil, the magnetic stimulus may be imposed not only on the target, but also on the adjacent areas of the brain thereabout. Correct alignment of the coil presumes a measurement of the head position of the patient being examined and/or treated.

In the prior art, the head of the patient being examined is adapted to be fixed and supported in place mechanically in a stereotactic frame of the prior-art type. The stereotactic frame must keep the patient's head immobilized during the examination and treatment procedures inasmuch as the head orientation and location are registered referenced to the coordinates of the mechanical fixtures of the stereotactic frame. An alternative technique of the prior art comprises a flexible stereotactic localizing band which is adapted attachable to the stereotactic frame and is mountable on the patient's head and, further, has mounted thereon and/or is capable of accommodating the attachment of fiducial marker elements thereon. In conventional localization frames, the fiducial markers are placed in a flexible band suited for attachment on the patient's forehead, for instance. Alternatively to or in parallel with the stereotactic frames, it is possible to use fiducial markers that can be adhered in a self-adhesive fashion to the skin of the patient being examined.

As the examined patient's head in this prior-art technology has to be mechanically fixed in place in the stereotactic frame, the head may not be allowed to move during the examination/treatment of the patient, because the position of the patient's head is registered in reference to the mechanical fixtures of the stereotactic frame. Such fixing to the head is most inconvenient to the patient and, during a session lasting several minutes, even the smallest changes permitted by the mechanical stereotactic fixture in the position of the patient's head may cause inaccuracies in the examination and/or treatment results. The mechanism used for fixing the head in place also sets limitations to the examination and/or treatment position of the patient. Due to the head fixing techniques used in conventional stereotactic frames, such operations as the examination and treatment of bedpatients in a lying position is impossible. Flexible headbands that are attachable to the patient's head and affixable to a stereotactic frame cause extreme inconvenience to the patient if they are tightened firmly on the head and yet are incapable of preventing fiducial markers from being displaced from their nominal position. In the use of conventional stereotactic frames, location shifts of fiducial markers cause significant dimensional errors. For instance, facial movements may cause dimensional deviations of up to several millimeters in the examination results. Flexible headbands are also difficult to place in exactly identical positions during successive examination sessions. Hence, the examination conditions are not repeatable from time to time. In a summary, the disadvantages of conventional stereotactic techniques include a position shift of fiducial markers during examination sessions and the resulting significant risk of localization inaccuracy error due such marker position shifts, inconvenience experienced by the patient due to the fixing technique of the stereotactic frame and the unfavorable location of the fiducial makers that disturbs the examination and/or treatment operations.

It is an object of the present invention to overcome the above-described problems of the prior-art techniques and to provide an entirely novel type of stereotactic frame.

The goal of the invention is achieved by virtue of adapting the stereotactic frame to rest with a substantial pressure on the nasion of the human or animal patient being examined and/or treated.

More specifically, the stereotactic frame according to the invention is characterized by what is stated in the characterizing part of claim 1. Furthermore, the method according to the invention for supporting a stereotactic frame is characterized by what is stated in the characterizing part of claim 13.

The stereotactic frame according to the invention offers substantial benefits. The stereotactic frame according to the invention is applicable to the localization of the position or positions of organs to be examined and/or treated. Furthermore, the location(s) of the organ(s) can be determined while the subject being examined is not immobilized. As a result, the stereotactic frame according to the invention is particularly suitable for use in medical applications. The head of the subject being examined need not be mechanically clamped in place during the use of the stereotactic frame according to the invention. Although the head position is registered in regard to the stereotactic frame, the head may be allowed to move during examination and/or treatment operations being performed. The fixation of the stereotactic frame to the subject's head is not inconvenient to the subject inasmuch as the subject's head may move freely without causing any essential inaccuracy in the results of the examination and/or treatment procedures. Furthermore, the stereotactic frame may move along with the head posture, whereby changes,in the head position do not cause essential measurement errors nor does the stereotactic frame supporting arrangement impose substantial limitations to the treatment posture of the patient being examined. The head of the subject being examined and/or treated need not be fixed. The stereotactic frame according to the invention, implemented in the form of a localizing bow for instance, permits the examination and treatment of, e.g., bed-patients in a lying position. A localizing bow affixable to the head is not experienced particularly inconvenient and need not be clamped tight about the head in order to prevent inadvertent movements of the fiducial markers. The movements of the fiducial markers do not cause essential measurement errors nor the subject's facial movements can cause deviations of several millimeters in the measurements. The localizing bow is uncomplicated to position with a relatively good accuracy always at the same point during different examination sessions. As a result, the examination conditions become repeatable with a reasonable accuracy.

Furthermore, certain embodiments of the localizing bow according to the invention can offer the following benefits. An embodiment of the stereotactic frame according to the invention is affixable so as to stay stationary irrespective of the facial movements of the subject being examined and/or treated. The stereotactic frame is adaptable to be supported to the upper portion of nasion that stays immobile during the facial movements of the patient being examined and/or treated. In this fashion, the patient being examined can be given the opportunity of talking during the examination, localization and/or treatment operations. The patient being examined and/or treated may use his/her own eyeglasses in conjunction with one embodiment of the stereotactic frame according to the invention. The stereotactic frame is convenient to wear by the patient inasmuch as there is no need to use any self-adhesive pads or a tightly adjustable headband, because the stereotactic frame may be adapted to support to the patient's nasion via a resiliently deforming surface that inherently assumes a shape compliant with the patient's nasion. The compliant surface may be selected to be of a resilient material such as expanded cellular PE plastic. A preferred embodiment of the stereotactic frame according to the invention comprises a localizing bow. The stereotactic frame designed, e.g., as a localizing bow, may include an elastic band adapted to pass behind the head without needing firm tightening. The localizing bow may be designed to prevent the formation of superficial indents on the face such as those caused by conventional eyeglasses. A single stereotactic frame may be adapted to comply with nose contours of different size and shape with the help of a replaceable nose piece of an elastic material such as cellular PE plastic or the like that complies with the individual shape of a nose. Advantageously, the adjustable elastic band acting as an essential element of the stereotactic frame and adapted to pass behind the patient's neck is made adjustable individually for each patient. The patients' hygiene can be assured by designing the stereotactic frame such that it can accommodate a nose piece which is easily replaceable for each patient. The patient head can be allowed to move freely during examination and/or treatment operations while the bow stays at the same time in place in a stable balanced position. The bow can be adapted to regain its initial position accurately even after strong swinging movements of the head. Hence, the localizing bow may be arranged if so desired not to interfere with the examination and/or treatment operations being carried out in conjunction with the localization steps inasmuch as the bow can be adapted frontally below the patient's eyes, whereby no long supporting arms extending like normal eyeglass bends from the sides of the head backward behind the ears are required. Respectively, the localizing bow need not be situated so as to obstruct the visual field of the patient being examined and/or treated. The stereotactic frame according to the invention permits free positioning and movement of the subject being located during the localization step. Hence, the stereotactic frame can be used in a patient-friendly fashion due to its easy and uncomplicated attachment. The stereotactic frame may be designed well fitting for any shape of the patient's nose and head. The stereotactic frame may be designed to resume its initial state after a deviation after the position of the bow has been deviated by touching the distal portions of the bow, for instance. Hence, swings and jerks imposed on the bow cannot cause essential inaccuracy in its position. The stereotactic frame can be adapted to be situated outside the patient's field of vision thus facilitating the patient to wear his/her eyeglasses in conjunction with the use of the stereotactic frame. This is particularly vital in situations, wherein the subject is assumed to possess an acute reaction capability such as is required in visual evoked response tests. The stereotactic frame may be designed to retain its shape during use, whereby no essential measurement inaccuracy will be caused by deformation of the localizing bow. The nasion portion of the stereotactic frame may be designed to incorporate a replaceable, personal nose piece that facilitates an economic means of providing the subject's personal hygiene through giving a new nose piece during each session for the subject being examined. Hence, the stereotactic frame according to the invention may or may not incorporate a replaceable nose piece. In an embodiment of the stereotactic frame according to the invention, the frame can be secured to the patient's head without the need for firmly tightening the elastic band that is passed behind the patient's head. Furthermore, the localizing bow according to the invention needs no supporting rails extending like normal eyeglass bends from the sides of the head backward behind the ears. Respectively, the localizing bow need not cause an obstruction to the visual field of the patient being examined and/or treated. Moreover, the localizing bow due to its advantageous positioning will not disturb operation about the subject's head nor become concealed behind instruments surrounding the subject's head.

In the following, the invention will be examined with the help of exemplifying embodiments by making reference to the appended drawing, wherein

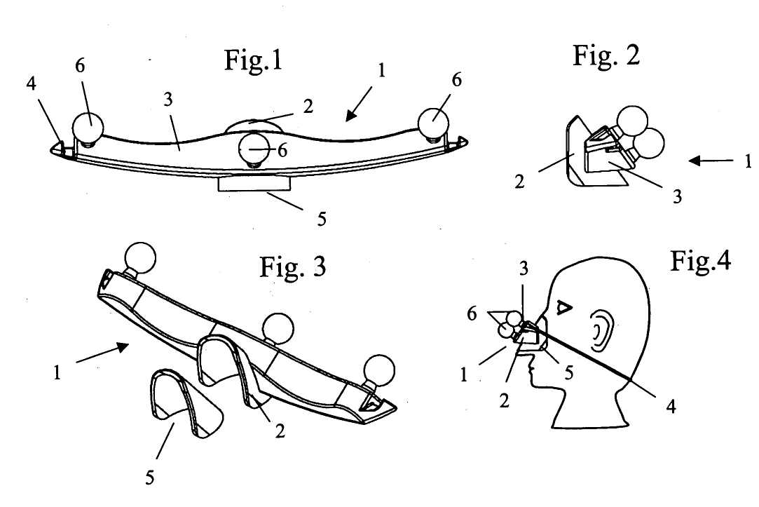

FIG. 1 shows a front view of an embodiment of the stereotactic frame according to the invention implemented as a localizing bow;

FIG. 2 shows a side elevation view of the same construction of the localizing bow;

FIG. 3 shows respectively an obliquely taken rear view of the same localizing bow, particularly the replaceable nose piece thereof; and

FIG. 4 shows a side view of supporting the localizing bow to the head of the subject being examined.

As shown in FIG. 1, a localizing bow 1 according to the invention is adapted to be supported to the middle of the nasion of the subject being examined and/or treated, which facial area is the sole area on the subject's face that stays unchanged even during the movements of facial muscles. The localizing bow 1 described below is able to eliminate the localization errors occurring in conjunction with the use of conventional stereotactic frames.

The localizing bow 1 comprises an adjustable band 4 adapted to pass behind the neck of the subject being examined, a frame 3 advantageously having a stiff structure and adapted to be outdistanced from the face, a nasion relator portion 2 shaped compliant with the nasion and a nose piece 5 replaceable on the nasion relator portion 2. By virtue of this construction and the nose piece 5, a single localizing bow 1 is suitable for use in conjunction with a substantially wide variety of different nose types and head shapes. The localizing bow 1 may also be used without the replaceable nose piece 5 or, alternatively, be at least designed useable without the replaceable nose piece 5. However, the nose piece 5, which advantageously is affixable to the nasion relator portion of the localizing bow by means of a self-adhesive tape and/or a shaped socket and complies with the subject's facial features, can substantially contribute to the user convenience of the localizing bow.

Due to its wavy structure, the localizing bow 1 can retain its stiff shape thus counteracting to errors resulting from the deformations of the stereotactic frame. The wavy shape of the device adapted to be situated below the eyes of the subject being examined and/or treated gives the subject a free field of vision. The supporting frame 3 having a triangular cross section is designed to retain the structural integrity of the localizing bow 1. The form of the localizing bow 1 facilitates placing fiducial markers 6 located on the exterior side of the localizing bow so as to have the markers outside the subject's field of vision. Advantageous positions for the fiducial markers 6 are the front side of the nasion relator portion 2 and the edges of the frame 3.

FIG. 2 shows a side view of the lateral contour of the exemplifying embodiment of localizing bow 1 illustrated in FIG. 1, whereby the side view elucidates a nasion relator portion 2 compliant with the nasion and a structurally stiff frame 3 situated outdistanced from the subject's face.

The side view of FIG. 3 showing the replaceable nose piece 5 of the nasion relator portion 2 of the localizing bow 1 illustrates one alternative of shaping and adapting the replaceable nose piece 5. The replaceable nose piece 5 is adapted to support on one side to the nasion relator portion 2 of the localizing bow 1 and, on the other side, to the middle of the nasion of the subject being examined and/or treated.

FIG. 4 shows in a side view an embodiment of the localizing bow according to the invention supported to the head of a subject or animal being examined and/or treated. This kind of stereotactic frame 1 is arranged to support an essentially entire compressive force to the subject or animal being examined and/or treated. However, an exception to such full-compression supporting to the subject may be made when, e.g., the subject being treated is a lying or sitting position. Herein, a portion of the stereotactic frame 1 may be arranged to support to the operating table or examination chair. The localizing bow comprises a support structure 2-4 adapted to support to the head of the subject being examined and/or treated. The support structure is generally referenced as a localizing bow 1. In detail, the localizing bow 1 comprises a nasion relator portion 2 adapted to support to the nasion, a frame 3 outdistanced from the subject's face and a band 4 that is adapted to pass behind the subject's neck and generally is supported below the ears. The nasion relator portion 2 of the localizing bow is shaped essentially compliant with the nasion, whereby the localizing bow can be adapted to provide enough room to accommodate the use of eyeglasses simultaneously therewith. The nasion relator portion 2 accepts a nose piece 5 adapted compliant with the subject's facial features so as to improve the stability of the localizing bow, increase user convenience and, in certain cases, to facilitate the use of a single localizing bow on noses of different sizes. The latter embodiment requires that the shape of the nose piece 5 is made variable. The affixing of the nose piece on the nasion relator portion of the localizing bow can be implemented using self-adhesive surfaces and/or a socketed joint. With the help of the replaceable nose piece 5, also the user hygiene of the localizing bow can be assured by using a new, virgin nose piece 5 of the localizing bow nasion relator portion separately for each subject being examined. Certain embodiments of the localizing bow 1 are provided with a band 4 adapted to pass behind the subject's head so as to support the localizing bow 1 to the head. The localizing bow 1 includes at least one fiducial marker 6 by means of which the position of the localizing bow relative to the organ being examined and/or treated can be defined. Advantageously, the frame 3 of the localizing bow 1 has such a structural shape that stiffens the localizing bow 1 against bending and, furthermore, prevents the frame 3 from obstructing the visual field of the object being examined and/or treated. Advantageously, the localizing bow 1 is placed frontally in regard to the subject's face so as to remain outside the subject's field of vision by virtue of not having any supporting rails extending along the sides of the subject's head in the same fashion as eyeglass bends. The localizing bow 1 does not interfere with operations about the subject's head and, due to its advantageous positioning, will not become concealed is behind instruments possibly placed about the subject's head.

Without departing from the scope and spirit of the invention, also alternative embodiments thereof may be contemplated.

By virtue of modifying the shape and/or size of the nose piece 5, the localizing bends 1 can be made to fit on noses of different sizes. For instance, a thicker nose piece 5 is advantageously selected for the smaller nasions of children and females as compared with the nose pieces suitable for the larger nasions of males. Thus, the localizing bow 1 can be supported to nasions of all sizes and shapes in an excellent fashion. Additional adaptivity to individual variations can be attained by selecting a nasion relator portion 2 suitable for the shape of the subject's nasion. For instance, the noses of Asian race subjects need a nasion relator portion shallower than that designed for the noses of European race subjects.

The stereotactic frame may alternatively comprise a number of elements supported to the object being examined so as to locate an organ being examined and/or treated.

In the context of the present application, the term “essential compressive force” must be understood to refer to such a compressive force that without additional support is sufficient to secure the stereotactic frame stable in place. In other words, a supporting force equal to the essential compressive force is sufficient to assure a positively secured position of the stereotactic frame.

An essential compressive force can be imposed with the help of an elastic neckband, for instance. Alternatively, the essential compressive force may be attained by affixing the frame with the help of an elastic element, e.g., behind the ears and/or by using a suitable design of the nose piece.

The invention can be utilized by way of first accurately measuring the position and orientation of the stereotactic frame relative to the subject's head with a localization device. First herein, to the subject's head is affixed a stereotactic frame having its position sensors set to measure the position and orientation of the head. The position of a sensor in regard to the fiducial marker points (at least 3 pcs.) of the head is determined by measuring the positions of the fiducial marker points with the help of the localization device. Next, the respective fiducial marker points are identified in magnetic-resonance images of the head, whereby a coordinate transformation is possible between the fiducial point locations of the position sensor and the points of the magnetic-resonance images. As a result, the fiducial marker element is arranged to define the location of the stereotactic frame and the organ being examined and/or treated relative to each other. Now, when the instrument(s) having another set of position sensors affixed thereon is/are placed in a close vicinity of the subject's head, the position and orientation of the instruments relative to the stereotactic frame can be defined accurately.

With the above-described coordinate transformation, the position and orientation of instruments can be determined in regard to any point selected from the magnetic-resonance images. The position sensor system may comprise, e.g., a group of IR-reflective balls. When an IR light pulse is launched toward the balls, the pulse is reflected from the balls. Using an appropriate high-resolution location measurement device, the reflected light rays can be detected so that the locations of the balls are identified. Having the group comprising at least three balls with, their mutual distance known, the exact position and orientation of the ball group can be determined. From the localization device, the position and orientation information of the position sensors is transmitted to a computer and shown in a proper format on a display, for instance.

1. A stereotactic frame (1) suitable for use in the determination of the position and/or orientation of a subject's head, characterized by
comprising at least three fiducial marker elements (6) for determining the location of said stereotactic frame (1), and
being adapted to support by an essential compressive force to the middle of the nasion of the subject being examined and/or treated so as to retain the stereotactic frame self-contained in place during an examination and/or treatment session.

2. The stereotactic frame of claim 1, characterized by comprising a supporting frame structure (2-4) supported to the head of the subject being examined and/or treated.

3. The stereotactic frame of any one of claims 1-2, characterized by having a localizing bow (1) in said frame structure.

4. The stereotactic frame (1) of any one of claims 1-3, characterized by including at least one nasion relator portion (2) adapted to support to the subject's nasion.

5. The stereotactic frame (1) of any one of claims 1-4, characterized by including a supporting body (3) outdistanced from the face of the subject being treated by virtue of being a supportive and/or supported member in relation to said nasion relator portion (2) and a supporting element (4).

6. The stereotactic frame (1) of any one of claims I-5, characterized in that said nasion relator portion (2) is shaped substantially compliant with the subject's nasion, whereby the design of the stereotactic frame (1) provides sufficient free space to accommodate the use of eyeglasses simultaneously with the stereotactic frame.

7. The stereotactic frame (1) of any one of claims 1-6, characterized in that said nasion relator portion (2) is adapted to provide support to at least one nose piece (5) compliant with the contour of the subject's face, whereby the nose piece (5) is either replaceable or has a modifiable shape or size such that allows the stability of the localizing bow to be improved, the user convenience to be enhanced and/or to facilitate the use of the localizing bow on another nasion of different size.

8. The stereotactic frame (1) of any one of claims 1-7, characterized by including a supporting element (4) such as a band adapted to pass behind the neck or other portion of the subject's head, or, alternatively, a supporting element (4) otherwise passed to supportingly the rear of the subject being examined and/or treated, e.g., is behind the subject's head, neck or shoulders.

9. The stereotactic frame (1) of any one of claims 1-8, characterized by being supportable to the external auditory channel and/or to the forehead, above the nasion.

10. The stereotactic frame (1) of any one of claims 1-9, characterized by including in the stereotactic frame (1) a band (4) passable behind the subject's head and/or neck so as to support the stereotactic frame to the head of the subject being examined and/or treated.

11. The stereotactic frame (1) of any one of claims 1-10, characterized by having the supporting body (3) of the stereotactic frame designed to structurally prevent the localizing bow from bending and/or shaped so as to leave the field of vision of the subject being examined and/or treated substantially unobstructed.

12. The stereotactic frame (1) of any one of claims 1-11, characterized by having the stereotactic frame (1) adapted placeable on the frontal side of the face of the subject being examined and/or treated so as to be situated substantially outside the subject's field of vision.

13. A method for supporting a stereotactic frame suitable for use in the localization of a subject's head, characterized in that the stereotactic frame (1) is supported with an essential compressive force to the middle of the nasion of the subject being examined and/or treated so as to retain the stereotactic frame (1) self-contained in place during an examination and/or treatment session, whereby the location of the stereotactic frame (1) is determined with the help of fiducial marker elements (6).

14. The method of claim 13 for supporting a stereotactic frame, characterized in that the stereotactic frame structure (2-4) is supported to the head of the subject being examined and/or treated.

15. The method of claim 14 for supporting a stereotactic frame, characterized in that said frame structure is a localizing bow (1).

16. The method of any one of foregoing claims 15-16 for supporting a stereotactic frame (1), characterized in that at least one nasion relator portion (2) of the stereotactic frame is supported to the nasion of the subject being examined and/or treated.

17. The method of any one of foregoing claims 15-17 for supporting a stereotactic frame (1), characterized in that therein is used a supporting body (3) outdistanced from the face of the subject being treated by virtue of the body being a supportive and/or supported member in relation to said nasion relator portion (2) and a supporting element (4).

18. The method of any one of foregoing claims 15-17 for supporting a stereotactic frame (1), characterized in that therein is used a nasion relator portion (2) shaped substantially compliant with the subject's nasion and/or that the use of eyeglasses simultaneously with the stereotactic frame (1) is facilitated.

19. The method of any one of foregoing claims 15-18 for supporting a stereotactic frame (1), characterized in that therein
support to said nasion relator portion (2) is provided by using at least one nose piece (5), and
the nose piece is made either replaceable or has a modifiable shape or size such that improves the stability of the localizing bow, enhances the user convenience and/or facilitates the use of the localizing bow on another nasion of different size.

20. The method of any one of foregoing claims 15-19 for supporting a stereotactic frame (1), characterized in that therein is used a supporting element (4) such as a band adapted to pass behind the neck or other portion of the subject's head, or, alternatively, a supporting element (4) otherwise passed supportingly to the rear of the subject being examined and/or treated, e.g., behind the subject's head, neck or shoulders.

21. The stereotactic frame (1) used in any one of claims 15-20, characterized by being adapted to be supported to the subject's external auditory channel and/or forehead, above the nasion.

22. The method of any one of foregoing claims 15-21 for supporting a stereotactic frame (1), characterized in that the stereotactic frame (1) is supported to the head of the subject being examined and/or treated by means of a band (4) adapted to pass behind the subject's head and/or neck.

23. The method of any one of foregoing claims 15-22 for supporting a stereotactic frame (1), characterized in that the position of the stereotactic frame relative to the organ being examined and/or treated is defined by means of at least one fiducial marker element included in the stereotactic frame (1).

24. The method of any one of foregoing claims 15-23 for supporting a stereotactic frame (1), characterized in that therein is used a supporting body (3) of the stereotactic frame, the supporting body being designed to structurally prevent the localizing bow from bending and/or being shaped so as to leave the field of vision of the subject being examined and/or treated substantially unobstructed.

25. The method of any one of foregoing claims 15-23 for supporting a stereotactic frame (1), characterized in that the stereotactic frame (1) is placed on the frontal side of the face of the subject being examined and/or treated so as to be situated substantially outside the subject's field of vision.
